(19) United States
(12) Patent Application Publication
(10) Pub. No.: US 2016/0151597 A1
(43) Pub. Date: Jun. 2, 2016

(54) BREATHING APPARTUS WITH NASAL INTERFACE

(71) Applicant: inSleep Technologies, LLC, Weston, FL (US)

(72) Inventors: Martin Baecke, Dessau-Roblau (DE); Marcel Borgward, Oedheim (DE); Michael H. Gusky, Weston, FL (US); Michael H. Lauk, Freiburg (DE); Kwame L. Osseo-Asare, Weston, FL (US)

(21) Appl. No.: 14/927,147

(22) Filed: Oct. 29, 2015

Related U.S. Application Data
(63) Continuation-in-part of application No. 14/662,798, filed on Mar. 19, 2015.
Application No. 14/662,798 is a continuation of application No. 13/280,650, filed on Oct. 25, 2011, now Pat. No. 8,985,115.
Application No. 13/280,650 is a continuation-in-part of application No. 13/425,049, filed on Mar. 20, 2012, now abandoned.
(60) Provisional application No. 61/406,315, filed on Oct. 25, 2010.
(60) Provisional application No. 61/467,760, filed on Mar. 25, 2011.

(51) Int. Cl. A61M 16/06; A61M 16/00

(52) U.S. Cl. CPC A61M 16/0666; A61M 16/0066; A61M 2205/3334

(57) ABSTRACT

An apparatus including a blower configured to provide a supply of breathing gas, and a delivery tube configured to deliver the supply of breathing gas to a user breathing interface. The delivery tube has an inside diameter of about 15 mm or less. The apparatus also including a control system configured to provide a control signal to the blower for controlling a pressure of the supply of breathing gas to between about 1 cm H₂O to about 6 cm H₂O at the user breathing interface. The control signal is based upon, at least in part, one of a pressure and a flow rate of the supply of breathing gas at the user breathing interface.

### CROSS-REFERENCE TO RELATED APPLICATIONS

This application is a Continuation-In-Part of, and claims priority to, co-pending U.S. patent application Ser. No. 14/662,798, entitled Nasal Interface and Removable Pad Therefor, filed Mar. 19, 2015, which itself is a continuation of U.S. patent application Ser. No. 13/280,650, entitled Butterfly Nasal Interface, filed Mar. 25, 2011, now issued U.S. Pat. No. 8,985,115, which claims the benefit of U.S. Provisional Patent Application Ser. No. 61/406,315, filed Oct. 25, 2010; and is also a Continuation-In-Part of, and claims priority to, co-pending U.S. patent application Ser. No. 13/425,049, entitled Breathing Apparatus, filed Mar. 20, 2012, which claims the benefit of U.S. Provisional Patent Application Ser. No. 61/467,760, filed on Mar. 25, 2011, the entire disclosures of which are incorporated by reference herein for all purposes and commonly owned.

### TECHNICAL FIELD

The present disclosure generally relates to a breathing apparatus, and more particularly relates to a breathing apparatus that may be used in connection with reducing snoring.

### BACKGROUND OF THE DISCLOSURE

Snoring is an affliction that affects many people. Snoring may be an ongoing, regular problem, or may occur intermittently or occasionally. Snoring may result in various problems, both to the person snoring as well as those around the person snoring, such as sleeping partners or cohabitants. For example, snoring has been linked to sleep deprivation, in which the sleeping patterns of the person snoring may be disrupted. Such sleep deprivation may result in daytime drowsiness, lack of focus, as well as other problems. Snoring can also be disruptive to those around the person snoring, similarly resulting sleep deprivation or disturbance of such people.

### SUMMARY OF THE DISCLOSURE

According to a first implementation, an apparatus may include a blower configured to provide a supply of breathing gas, and a delivery tube configured to deliver the supply of breathing gas to a user breathing interface. The delivery tube may have an inside diameter of about 15 mm or less. A control system may be configured to provide a control signal to the blower for controlling a pressure of the supply of breathing gas to between about 1 cm H₂O to about 6 cm H₂O at the user breathing interface. The control signal may be based upon, at least in part, one of a pressure and a flow rate of the supply of breathing gas at the user breathing interface.

One or more of the following features may be included. The blower may include a motor and an impeller. A speed of the motor may be controlled based upon, at least in part, the control signal. The blower may be configured to supply the breathing gas having a peak pressure of about 25 mbar at a flow rate of about 100 l/min at an outlet of the blower. The blower may be configured to supply the breathing gas having a pressure of about 30 mbar and a flow rate of about 0 l/min at an outlet of the blower. The blower may be configured to provide a flow rate acceleration of about 150 l/min/s over a flow rate range of about 0 l/min to about 100 l/min. The blower may be configured to provide the flow rate acceleration of about 150 1/min/s over a pressure range of from about 0 mbar to about 25 mbar.

The delivery tube may include a cross-sectional area adjacent the user breathing interface that is smaller than a cross-sectional area adjacent the blower. At least a portion of the delivery tube may include a corrugated configuration. At least a portion of the delivery tube may include an exterior profile having an at least partially flat surface.

The apparatus may further include a pressure sensor coupled with the user breathing interface. The pressure sensor may provide an output signal indicative of the pressure of the supply of breathing gas at the user breathing interface. The control signal of the control system may be based upon, at least in part, the output signal. The pressure sensor may be coupled with the user breathing interface via a measurement lumen fluidly coupled with the pressure sensor and the user breathing interface. The delivery tube may include a multi-lumen tube including the measurement lumen and a breathing gas delivery lumen. A wall between the measurement lumen and the breathing gas delivery lumen may be configured to de-couple pressure effects of the breathing gas in the breathing gas delivery lumen from the measurement lumen. The wall may include a region of increased thickness. The wall may include a region of increased hardness. The delivery tube may include an integrated multi-lumen connector. The multi-lumen connector may be configured to provide a rotationally symmetrical connection.

According to another implementation, an apparatus may include a blower assembly configured to provide a supply of breathing gas. A delivery tube may include a delivery lumen configured to deliver the supply of breathing gas to a user breathing interface, and a measurement lumen fluidly coupled to the user breathing interface. The delivery lumen may have an inside diameter of about 15 mm or less. A sensor may be fluidly coupled to the measurement lumen. The sensor may be configured to measure at least one of a pressure of breathing gas at the user breathing interface and a flow rate of breathing gas at the user breathing interface. A controller may be coupled to the blower for controlling an output characteristic of the breathing gas, based upon, at least in part, a measurement signal received from the sensor for controlling a pressure at the user breathing interface to between about 1 cm H₂O to about 6 cm H₂O.

One or more of the following features may be included. A cross-sectional area of the delivery tube adjacent the blower assembly may be greater than a cross-sectional area of the delivery tube adjacent the user breathing interface. The measurement lumen of the delivery tube may be configured to decouple the measurement lumen from pressure effects of the delivery lumen.

The blower assembly may have an acceleration of about 150 l/min/s over a flow rate from about 0 l /min to about 100 l/min.

The apparatus may further include a multi-lumen connector configured to couple the delivery lumen with the blower assembly and the measurement lumen with the sensor. The multi-lumen connector may provide a rotationally symmetrical connection.

According to another implementation, an apparatus may include a housing assembly including a blower configured to provide a supply of breathing gas. A user breathing interface may be configured to fluidly couple with an airway of a user. A supply tube may be configured to fluidly couple the blower with the user breathing interface. The supply tube may have an inside diameter of between about 15 mm to about 5 mm. A control system may control the blower to provide a breathing gas pressure of between about 1 cm H₂O to about 6 cm H₂O at the user breathing interface.

One or more of the following features may be included. The control system may include as pressure sensor fluidly coupled with the user breathing interface. The supply tube may include a delivery lumen configured to fluidly couple the blower with the user breathing interface, and a measurement lumen configured to fluidly couple the sensor with the user breathing interface. The blower may include a motor and an impeller. A speed of the motor may be controlled based upon, at least in part, a control signal from the control system.

According to yet another implementation, a nasal interface can comprise a base portion having a least one inlet; a soft pad portion, including a lower border operatively connected to the base portion, wing portions positioned at an angle relative to each other and having at least one hole formed therein, and a bellows portion connecting the lower border and the wing portions.

The details of one or more implementations are set forth in the accompanying drawings and the description below. Other features will become apparent from the description, the drawings, and the claims.

### BRIEF DESCRIPTION OF THE SEVERAL VIEWS OF THE DRAWINGS

FIG. 1 schematically depicts a breathing apparatus consistent with an illustrative embodiment of the present disclosure.

FIG. 2 is cross-sectional view of an embodiment of a multi-lumen delivery tube that may be used in connection with the breathing apparatus shown in FIG. 1.

FIG. 3 diagrammatically depicts an embodiment of a multi-lumen connector that may be used in connection with the breathing apparatus of FIG. 1.

FIG. 4 diagrammatically depicts another embodiment of a multi-lumen connector that may be used in connection with the breathing apparatus of FIG. 1.

FIG. 5 diagrammatically depicts an embodiment of a delivery tube that may be used in connection with the breathing apparatus of FIG. 1.

FIG. 6 diagrammatically depicts an embodiment of a housing including one or more display features and user control features that may be used in connection with the breathing apparatus of FIG. 1.

FIG. 7 schematically depicts an embodiment of a feedback control system that may be used in connection with the breathing apparatus of FIG. 1.

FIG. 8 schematically depicts an embodiment of a sensor and control system that may be used in connection with the breathing apparatus of FIG. 1.

FIG. 9 schematically depicts an embodiment of a modular control system that may be used in connection with the breathing apparatus of FIG. 1.

FIG. 10 diagrammatically depicts an exploded view of an embodiment of a breathing apparatus of FIG. 1.

FIG. 11 is a schematic view of a general system that can employ a nasal interface in accordance with the present disclosure.

FIG. 12 is a right rear perspective view of an illustrative embodiment of the present disclosure.

FIG. 13 is a front elevational view of the illustrative embodiment of the present disclosure shown in FIG. 12.

FIG. 14 is a cross-sectional view of the illustrative embodiment show in FIGS. 12 and 13 taken along line 4-4 of FIG. 16.

FIG. 15 is a top plan view of the base portion in accordance with an embodiment of the disclosure.

FIG. 16 is a top plan view of a soft pad portion of a nasal interface in accordance with the present disclosure.

FIG. 17A is a cross-sectional view of a nasal interface in accordance with the present disclosure taken along line 7A-7A of FIG. 16.

FIG. 17B is a left side elevational view of a nasal interface in accordance with the present disclosure.

FIG. 18 illustrates a user wearing a nasal interface in accordance with the present disclosure.

FIG. 19 illustrates a user wearing a nasal interface in accordance with the present disclosure.

### DETAILED DESCRIPTION OF PREFERRED EMBODIMENTS

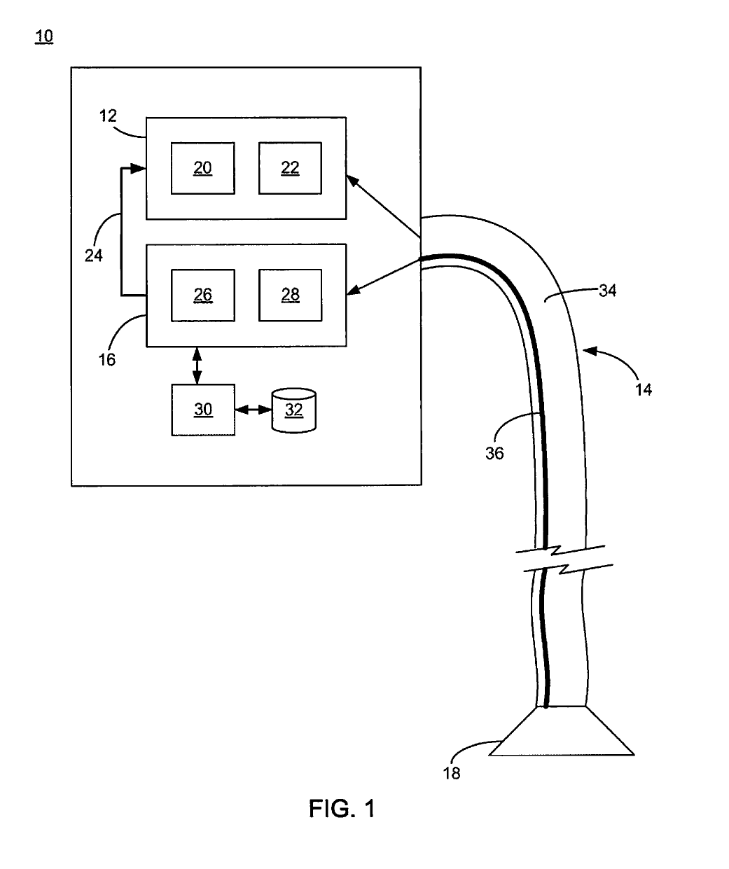

Referring to FIG. 1, an embodiment of breathing apparatus 10 is schematically shown generally including blower 12, delivery tube 14, and control system 16. Delivery tube 14 of breathing apparatus 10 may have an inside diameter that is about 15 mm or less, and may be configured to be fluidly coupled to user breathing interface 18. User breathing interface 18 may be configured to at least partially sealing engage one or more of a nasal passage of the user and the mouth of the user. Control system 16 may be configured to provide a control signal to blower 12 for controlling a pressure of the supply of breathing gas to between about 1 cm H₂O to about 6 cm H₂O at user breathing interface 18. In some embodiments, breathing apparatus 10 may be configured for use in connection with controlling or mitigating snoring of a user by providing positive upper airway pressure. The positive upper airway pressure provided by blower for the control and/or mitigation of snoring may generally be about 6 cm H₂O or less.

Consistent with the foregoing, an embodiment of a breathing apparatus of the present disclosure may provide positive upper airway pressure that be utilized to control and/or mitigate the occurrence of snoring in some users. The breathing apparatus may provide a generally constant positive upper airway pressure (e.g., as measured by pressure at the user breathing interface) in the range of between about 6 cm H₂O and 1 cm H₂O throughout a breathing cycle of the user. Various additional/alternative pressure ranges may also be utilized. For example, the breathing apparatus may provide a generally constant positive upper airway pressure that may be in the range from about 2 cm H₂O to about 6 cm H₂O, and/or in the range from about 2 cm H₂O to about 4 cm H₂O. Suitable positive upper airway pressures utilized in connection with a breathing apparatus consistent with the present disclosure may include any pressures within the above-discussed ranges.

Additionally, as discussed above, an embodiment of a breathing apparatus may include a delivery tube coupling an output of the blower with the user breathing interface having an inside diameter of about 15 mm or less. In some embodiments, the delivery tube may have an inside diameter of between about 5 mm and about 15 mm. Consistent with the present disclosure the delivery tube may have any cross-sectional geometry including, but not limited to, circular, oval, D-shaped, and polygonal. Reference herein to an inside diameter of the delivery tube may be applied to cross-sectional geometries other than circular by analogous cross-sectional area as compared to a circular cross-section delivery tube. For example, an oval delivery tube having a cross-sectional area equivalent to the cross-sectional area of a circular delivery tube having an inside diameter of 15 mm may be considered a delivery tube having an inside diameter of 15 mm. Consistent with an embodiment, a delivery tube having an inside diameter of about 15 mm or less may provide an improved user comfort and convenience of user as compared to larger diameter delivery tubes conventionally utilized in connection with breathing devices utilized for treating obstructive sleep apnea. For example, delivery tubes consistent with the present disclosure may allow less restricted movement of the user, e.g., through greater flexibility and reduced bulk.

Various user breathing interfaces may be used in connection with a breathing apparatus of the present disclosure. For example, the user breathing interface may include a full-face mask, which may sealing engage both the mouth and the nose of the user. Other user breathing interfaces may be configured to only sealingly engage the nasal passages of the user, e.g., via nasal pillows and/or prongs that may sealingly engage the nares of the user. Examples of user breathing interfaces that may suitably be utilized in connection with the breathing apparatus of the present disclosure are may include user breathing interfaces shown and described in one or more of US patent application Ser. No. 12/762,633, entitled Breathing Apparatus, filed on 19 Apr. 1010; U.S. patent application Ser. No. 61/406,315, entitled Nasal Interface, filed on 25 Oct. 2010; U.S. patent application Ser. No. 61/410,134, entitled Breathing Apparatus, filed on 4 Nov. 2010; U.S. patent application Ser. No. 61/423,195, entitled Tubing and Fixation of a Nasal Interface to Deliver Breathing Gases, filed on 15 Dec. 2010; and U.S. patent application 61/501,444, entitled Nasal Interface, filed on 27 Jun. 2011, the entire disclosures of all of which applications are incorporated herein by reference.

In some embodiments, blower 12 may include electric motor 20 coupled for driving impeller 22 (e.g., a centrifugal impeller). Blower 12 may provide a supply of breathing gas, e.g., for the generation of positive airway pressure at the user breathing interface. The supply of breathing gas may include air pressurized by impeller 22, e.g., to be delivered to user breathing interface 18 via delivery tube 14. In other embodiments, blower 12 may include various other systems for providing a supply of breathing gas, for example, a positive displacement air pump, a diaphragm pump, or the like. Further, in addition to air, the supply of breathing gas may be augmented and/or supplemented with other breathing gasses, such as oxygen. Such other breathing gasses may be provided from a suitable source, such as a container of pressurized gas.

As mentioned above, control system 16 may provide a control signal (e.g., control signal 24) to blower 12 for controlling a pressure of the supply of breathing gas at user breathing interface 18 to between about 1 cm H₂O to about 6 cm H₂O. According to an embodiment, control signal 24 may control a speed of motor 20 to thereby control a pressure and/or flow rate of the supply of breathing gas provided by blower 12. In other embodiments, control signal 24 may control the supply of breathing gas generated by blower 12 through other suitable mechanisms, such as varying a blower output nozzle characteristic, a flow restriction associated with blower 12, an exhaust or bypass valve, or the like.

The pressure and/or flow rate of the supply of breathing gas provided by blower 12 may vary over time, for example with a breathing cycle of the user, to maintain a generally constant pressure at user breathing interface 18 over the course of the breathing cycle of the user. For example, in an embodiment control system 16 may control a pressure of the supply of breathing gas at user breathing interface 18 to be generally constant throughout the breathing cycle of the user. As such, during inhalation of the user an output pressure and/or flow rate of the supply of breathing gas provided by blower 12 may be increased to provide a desired pressure of the supply of breathing gas at user breathing interface 18. Correspondingly, during exhalation of the user an output pressure and/or flow rate of the supply of breathing gas provided by blower 12 may be decreased to provide the desired pressure of the supply of breathing gas at user breathing interface 18.

Consistent with an embodiment, blower 12 may be configured to supply the breathing gas having a peak output pressure of about 25 mbar at a flow rate of about 100 l/min at an output of the blower, e.g., which may be experienced during an inhalation breathing cycle. In such an embodiment, a peak pressure of about 25 mbar at a flow rate of about 100 l/min may accommodate an inhalation segment of the breathing cycle of the user. Of course, the peak pressure and flow rate provided by blower 12 may vary depending upon the requirements of the user. For example, a relatively large adult user have a relatively larger lung capacity may require a greater blower output pressure and/or output flow rate than may be required by a user having a relatively smaller lung capacity, such as a child or adolescent. As will be discussed in greater detail below, an output pressure and/or flow rate of blower 12 may be greater than a desired pressure and/or flow rate at user breathing interface 18 due to a pressure drop associated with delivery tube 14.

Blower 12 may also be configured to provide a relatively high pressure at a relatively low flow rate, for example, during an exhalation breathing cycle of the user. For example, blower 12 may be configured to supply the breathing gas having a pressure of about 30 mbar at a flow rate of about 0 l/min at the outlet of blower 12. Consistent with such an example, the relatively high pressure and relatively low flow rate at the outlet of blower 12 may aid in controlling the occurrence of carbon dioxide, present accumulating in delivery tube 14. For example, user breathing interface 18, and/or a portion of deliver tube 14 adjacent user breathing interface 18 may include an exhaust valve or port configured to allow the escape of exhaled breath from user breathing interface 18 and/or a portion of delivery tube adjacent user breathing interface 18. The relatively high pressure and low flow rat may provide a residual pressure within delivery tube 14 to reduce and/or minimize the flow of exhaled breath into delivery tube 14, and thereby allow the exhaled breath to be preferentially exhausted out of the exhaust valve or port.

In some situations, the change over of the breathing cycle of the user from exhalation to inhalation may be relatively rapid, e.g., from a relatively low output demand on breathing apparatus 10 during an exhalation segment of the breathing cycle to a relatively high output demand on breathing apparatus 10 as the user begins an inhalation segment of the breathing cycle. According to an embodiment, blower 12 may have a relatively rapid acceleration to accommodate the change of breathing cycle segments (e.g., between inhalation and exhalation) without providing a user sensation of either overpressure (e.g., a sensation or resistance to exhaling) or a user sensation of under-pressure (e.g., a sensation of inadequate air during inhalation). For example, blower 12 may be configured to provide a flow rate acceleration of about 150 l/min/s over a flow rate range of about 0 l/min to about 100 l/min at an outlet of the blower. Further, blower 12 may be configured to provide a flow rate acceleration of about 150 l/min/s over a pressure range of about 0 mbar to about 25 mbar at an outlet of the blower. The foregoing flow rate acceleration, flow rate range, and pressure range is provided herein consistent with one embodiment. However, other flow rate accelerations, flow rate ranges, and pressure ranges may vary depending upon design criteria and user need.

According to one embodiment, control system 16 may include controller 26, which may provide control signal 24 to blower 12. Controller 26 may provide control signal 24 based upon, at least in part one or more sensory inputs (e.g., provided by sensor 28). In such an embodiment, controller 26 may include a suitable feedback controller. Examples of such feedback controllers may include a proportional-integration controller (PI controller), proportional-integration-derivative controller (PID controller), and/or other suitable controllers.

Sensor 28 (e.g., which may provide one or more sensory inputs to controller 26) may include one or more sensors configured to provide a sensor output based upon one or more characteristics of the supply of breathing gas (e.g., pressure and/or flow rate, etc.) at user breathing interface 18 and/or at the output of blower 12. Additionally/alternatively, sensor 28 may include one or more sensors configured to provide a sensor output based upon one or more user characteristics, such as an indicator of snoring, a user oxygen saturation, carbon dioxide level (e.g., within user breathing interface 18 and/or a portion of delivery tube 14 adjacent user breathing interface 18), an electrophysiological characteristic of the user, and the like. Control signal 24 to blower 12 may be based upon, at least in part, one or more of the sensor outputs. Additionally/alternatively, one or more sensor outputs may be received by a processors (e.g., processor 30) and/or stored by a computer readable medium (e.g., computer readable medium 32), examples of which may include, but are not limited to, a flash memory, a hard disk drive, a solid state disk drive, and a random access memory (e.g., RAM). Such stored sensor outputs may be utilized, for example, for providing diagnosis of user conditions, and/or monitoring of user conditions.

In one embodiment, sensor 28 may include a pressure sensor that may be coupled with user breathing interface 18 for providing a sensor output indicative of a pressure of the supply of breathing gas at, and/or within, user breathing interface 18 (e.g., which may be the same as and/or correlated to a pressure within an upper airway of the user). Accordingly, controller 26 of control system 16 may provide control signal 24 to blower 12 based upon, at least in part, the output signal of sensor 28. In an embodiment in which controller 26 may include a feedback controller, control system 16 may provide control signal 24 for controlling blower 12 to maintain a generally constant pressure of the breathing gas at user breathing interface 18 (and, therefore a generally constant pressure in the upper airways of the user) throughout the breathing cycle based upon, at least in part, changes in pressure at user breathing interface 18 detected by sensor 28.

In one embodiment, sensor 28 (e.g., which may include a pressure sensor) may be coupled with user breathing interface 18 via a measurement lumen fluidly coupled with pressure sensor 28 and user breathing interface 18. In one embodiment, delivery tube 14 may include a multi-lumen tube, in which one lumen may include breathing gas delivery lumen 34, and another lumen may include measurement lumen 36. Measurement lumen 36 may, in some embodiments, have an inside diameter of about 2 mm, although other diameter may also be suitable utilized for coupling sensor 28 with user breathing interface 18. In such an embodiment, breathing gas delivery lumen 34 may fluidly coupled blower 12 (e.g., an outlet of blower 12) with user breathing interface 18 for providing the source of breathing gas to user breathing interface 18 for user respiration. Measurement lumen 36 may fluidly couple user breathing interface 18 with sensor 28. Accordingly, sensor 28 may measure a pressure within measurement lumen 36, e.g., which pressure may be indicative of a pressure within user breathing interface 18 and/or may be correlated with a pressure within user breathing interface 18. In addition/as an alternative to a multi-lumen delivery tube, the measurement lumen may include a lumen separate from delivery tube 14, e.g., in a form of a measurement tube. In such an embodiment, the measurement tube may be separate from and/or coupled to delivery tube 14. In a further embodiment, sensor 28 may be disposed at least partially within, and/or adjacent to, user breathing interface 18 and may be electrically coupled with controller 26, e.g., via one or more electrical connections that may be integrated within and/or associated with delivery tube 14.

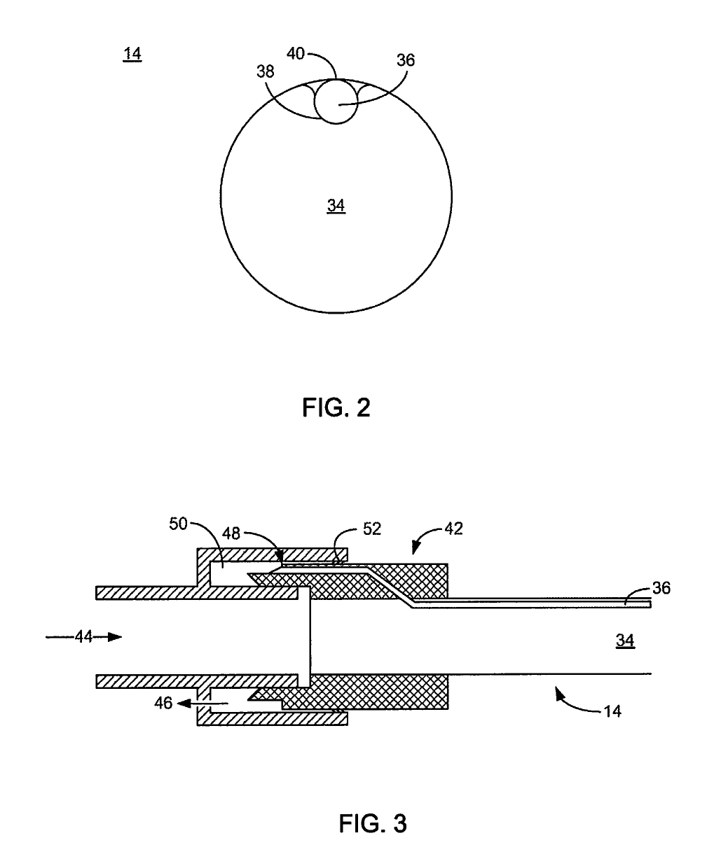

Referring also to FIG. 2, in an embodiment in which delivery tube 14 may include a multi-lumen tube, a wall (e.g., wall 38) of delivery tube 14 between measurement lumen 36 and breathing gas delivery lumen 34 may be configured to decouple pressure effects of the breathing gas in breathing gas delivery lumen 34 from measurement lumen 36. For example, as mentioned above, delivery tube 14 may have an inside diameter of about 15 mm or less. As such, delivery tube 14 may have a relatively large associated pressure drop. Accordingly, achieving a desired pressure at user breathing interface 18 may require providing the supply of breathing gas at the blower outlet (e.g., providing the supply of breathing gas to delivery lumen 34 at the blower outlet) having a relatively higher pressure. The relatively higher pressure adjacent the blower outlet may impart a pressure on measurement lumen 36. Consistent with an embodiment, a wall of delivery tube 14 between measurement lumen 36 and breathing gas delivery lumen 34 may be configured to decouple the pressure effects of the relatively higher pressure within breathing gas delivery lumen 34 adjacent the blower outlet as compared with the pressure within breathing gas delivery lumen adjacent user breathing interface 18.

According to one embodiment wall 38 between breathing gas supply lumen 34 and measurement lumen 36 may be configured to decouple pressure effects breathing gas within breathing gas delivery lumen 34 from measurement lumen 36 by including a region of increased thickness. For example, the region of increased thickness may include a region of wall 38 separating measurement lumen 36 from breathing gas delivery lumen 34. In some embodiments, the region of wall 38 separating measurement lumen 36 from breathing gas delivery lumen 34 may have a thickness greater than a thickness of wall 40 separating measurement lumen 36 from an exterior of delivery tube 14. Further, in some embodiments the thickness of wall 38 may vary about the length of delivery tube 14. For example, a thickness of wall 38 adjacent blower 12 may be greater than a thickness of wall 38 adjacent user breathing interface 18. In still further embodiments, the thickness of wall 38 may be generally constant about the length of delivery tube 14 and/or may be generally the same as the thickness of wall 40. In such an embodiment, the thickness of wall 38 may be configured to reduce and/or minimize pressure effects of breathing gas within breathing gas delivery lumen 34 from measurement lumen.

In addition/as an alternative to wall 38 having a thickness configured to decouple pressure effects of breathing gas within breathing gas delivery lumen 34 from measurement lumen 36, wall 38 may include a material having a hardness configured to decouple pressure effects of breathing gas within breathing gas delivery lumen 34 from measurement lumen 36. For example, the material of wall 38 may have a hardness that may resist and/or reduce deflect and/or deformation of wall 38 under the pressure of breathing gas within breathing gas delivery lumen 34.

Referring also to FIG. 3, in an embodiment in which delivery tube 14 includes a multi-lumen tube, and/or an embodiment in which the measurement lumen may include a separate tube that may be bundled with and/or coupled to delivery tube 14, delivery tube 14 may include an integrated multi-lumen connector (e.g., multi-lumen connector 42). As shown in the illustrative embodiment of FIG. 3, connector 42 may be configured to provide a rotationally symmetrical connection. That is, connector 42 may be configured to couple breathing gas delivery lumen 34 with a source of breathing gas (e.g., breathing gas source 44, which may include and/or be coupled with the outlet of blower 12) and couple measurement lumen 36 with a measurement port (e.g., measurement port 46, which may include and/or be coupled with sensor 28) in any rotational orientation. As such, in operation it may be unnecessary to achieve a particular rotational of multi-lumen connector 42 relative to mating connector 48 (e.g., which may include breathing gas source 44 and/or measurement port 46), thereby providing facile connection of delivery tube 14 with breathing apparatus 10. Further, the rotationally symmetrical configuration of the connector may allow the connector to swivel or twist while still maintaining a sealed connection. In such an embodiment, various detents, or other catches, may maintain the sealed connection and/or reduce the occurrence of disconnection, e.g., relative to a friction-fit engagement.

As shown in FIG. 3, in one embodiment, connector 42 may include a generally annular measurement coupling 48 that may be at least partially received in cooperating annular recess 50. Generally annular measurement coupling 48 may be fluidly coupled with measurement lumen 36 of delivery tube 14. Cooperating annular recess 50 may be coupled with measurement port 46. Breathing gas lumen 34 may be arranged generally coaxially with annular measurement coupling 48. Accordingly, when annular measurement coupling 48 is at least partially received in cooperating annular recess 50, breathing gas lumen 34 may be correspondingly coupled with breathing gas source 44. Consistent with the illustrated embodiment, one or more sealing features (e.g., sealing lips 52) may be included for sealing one or more of annular measurement coupling 48 with cooperating annular recess 50 and/or breathing gas lumen 34 with breathing gas source 44.

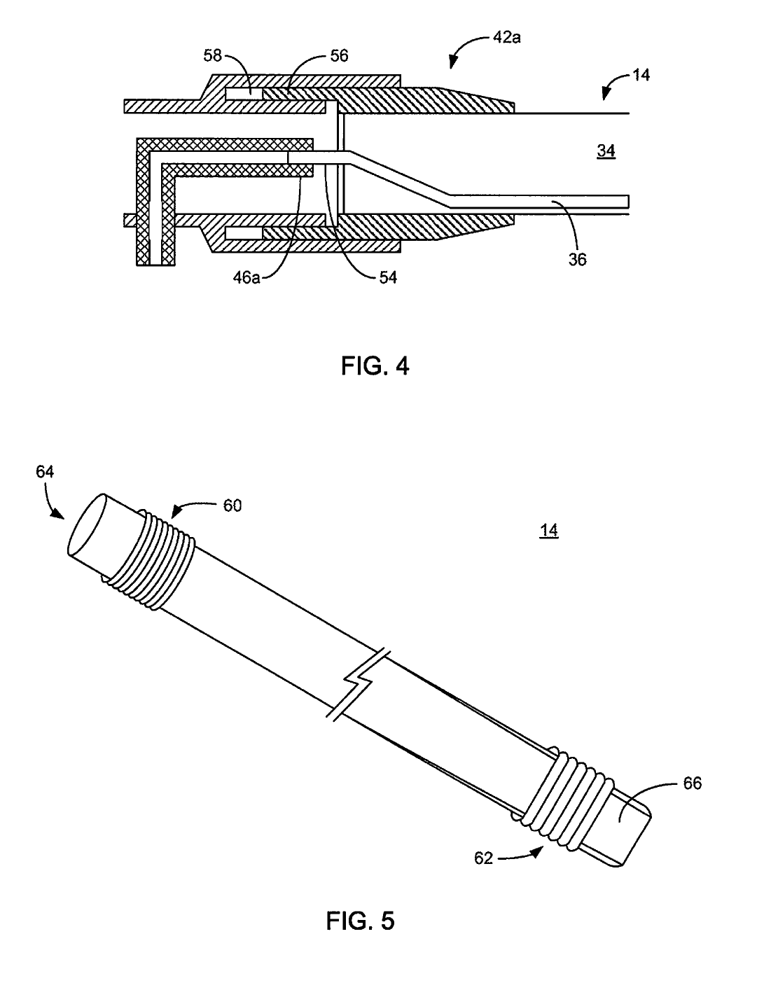

Referring also to FIG. 4, in a related embodiment connector 42a may include measurement lumen stem 54 configured to sealingly engage measurement port 46a. Measurement lumen stem 54 may be fluidly coupled with measurement lumen 36 of delivery tube 14. For example, at least a portion of measurement lumen stem 54 may be at least partially received within measurement port 46a and/or at least a portion of measurement port 46a may be at least partially received within measurement stem 54. Further, connector 42a may include generally annular engagement feature 56 that may be at least partially received within cooperating annular recess 58. Generally annular engagement feature 56 may be fluidly coupled with breathing gas delivery lumen 34 of delivery tube 14. As shown, measurement lumen stem 54 may be generally coaxial with annular engagement feature 56. Accordingly, measurement lumen stem 54 may couple with measurement port 46a in any rotational orientation of connector 42a. While not separately indicated one or more of measurement lumen stem 54, measurement port 46a, annular engagement feature 56 and cooperating annular recess 58 may include sealing features that may at least partially seal the respective components relative to one another and/or relative to an ambient atmosphere.

Various additional/alternative multi-lumen connector configurations may similarly be implemented, which may provide a rotationally symmetrical connection, may also be implemented. Further, while the foregoing illustrative embodiments of multi-lumen connectors have been generally discussed in the context of a connector that may be utilized between the delivery tube an the blower and/or sensor, in some embodiments a similar multi-lumen connector may be utilized between the delivery tube an the user breathing interface.

In some embodiments, delivery tube 14 may be generally tapered in diameter about the length of delivery tube 14, and/or may include a tapered region resulting in a decrease in diameter of delivery tube 14. Accordingly, in some embodiments, delivery tube 14 may include a cross-sectional area adjacent user breathing interface 18 that may be smaller than a cross-sectional area of delivery tube 14 adjacent blower 12. A reduced cross-sectional area adjacent user breathing interface 18 may, in some embodiments, improve user comfort, e.g., reducing the bulk, of delivery tube 14 adjacent the user and/or be decreasing restrictions on user movement.

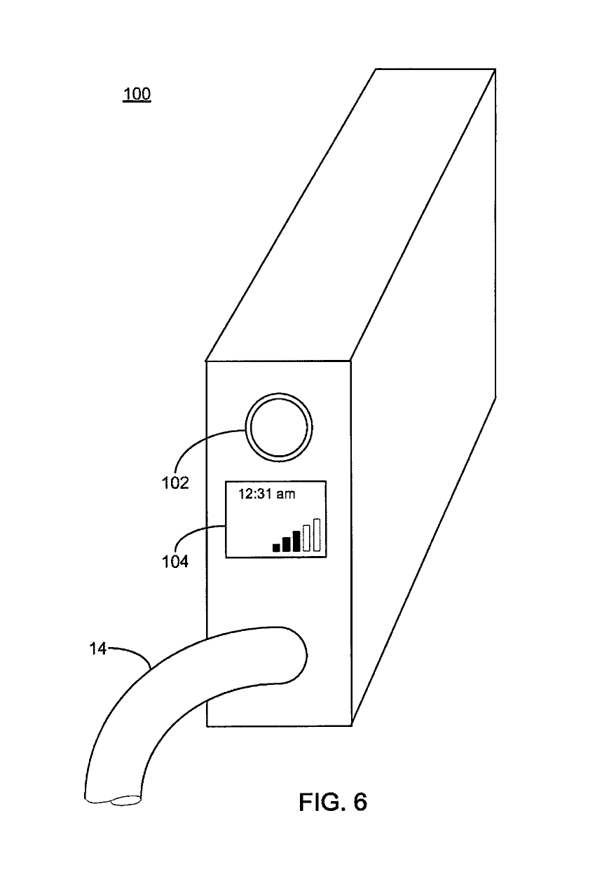

Referring also to FIG. 5, an illustrative embodiment of delivery tube 14 is shown in which at least a portion of delivery tube 14 may include a generally corrugated configuration. In the illustrated embodiment, delivery tube 14 is shown including a generally corrugated configuration (e.g., corrugated regions 60, 62) adjacent either end of delivery tube 14. In various additional/alternative embodiments the delivery tube may other corrugated configurations. For example, the delivery tube may be corrugated about a substantial portion of the length of the delivery tube. Further, the delivery tube may include only a single corrugated region adjacent a single end of the delivery tube, or a single corrugated region generally centrally about the length of the delivery tube. Various additional/alternative corrugated configuration may also be implemented.

In various embodiments, the corrugated configuration of delivery tube 14 may improve the flexibility of delivery tube 14, e.g., by providing increased flexibility of delivery tube 14 in the corrugated regions. Additionally/alternatively the corrugated configuration of delivery tube 14 may improve the crush and/or kink resistance of delivery tube 14, at least in the corrugated region(s) thereof. The corrugated configuration of delivery tube 14 may include various configurations, such as generally helical corrugations, linearly spaced corrugations, and the like, depending upon design criteria and preference. For example, in an embodiment, the delivery tube may include a helical corrugation member that may be generally coupled with a multi-lumen inner-delivery tube. In such an embodiment, the multi-lumen inner-delivery tube may include the breathing gas delivery lumen and the measurement lumen. The multi-lumen inner-delivery tube may be formed having relatively thin walls, and the helical corrugation member may provide a desired degree of crush and/or kind resistance to the delivery tube.

Still referring to FIG. 5, in some embodiments, at least a portion of delivery tube 14 may include a cross-sectional shape that may be different from a cross-sectional shape of at least another portion of delivery tube 14. For example, in the illustrated embodiment, delivery tube may include first portion 64 that may have a generally circular cross-sectional shape. Delivery tube 14 may further include at least second portion 66 having an at least partially flat surface. According to various additional/alternative embodiments the delivery tube may include various portions having different cross-sectional shapes, for example, oval, round, polygonal and the like.

Referring to FIG. 6, housing 100 is generally depicted. Housing 100 may be configured to at least partially contain one or more of blower 12 and control system 16. Housing 100 may include various internal features, such as elastomeric and/or viscous mounts for blower 12, e.g., which may reduce and/or minimize noise and/or vibration resulting from the operation of blower 12. Further, housing 100 may include one or more filter panels for filtering an air intake of blower 12. According to an embodiment, the one or more filter panels may include relatively large filter panels, e.g., which may minimize a pressure drop associated with the filter panel.

As shown in FIG. 6, housing 100 may also include various user control and/or display features. For example, housing 100 may include combined push-turn control 102. Push-turn control 102 may generally be provided as a rotary control knob that may be rotated to provide a control signal, e.g., to increase the relative pressure at the user breathing interface (e.g., as may be necessary to achieve a desired level of snoring mitigation), or to provide another control input. In one such embodiment, push-turn control 102 may provide a tactile feedback responsive to rotation of push-turn control 102. For example, push-turn control 102 may provide a click-type tactile feedback in response to rotation of push-turn control 102. In addition to providing a rotary control input, push-turn control 102 may be depressed to provide another control input (such as a select, mode, option change or other input). In one embodiment, the entirety of push-turn control 102 may be depressed. In another embodiment, push-turn control 102 may include an outer rotary bezel that may be rotated to provide a rotary control signal and an inner push button that may be depressed without depressing the outer rotary bezel.

Additionally, housing 100 may include one or more information displays (e.g., display 104), e.g., liquid crystal displays, organic light emitting diode displays, or the like. Display 104 may display various information relative to the operation and/or settings of breathing apparatus 10. For example, as shown display 104 may provide an indicator of relative pressure at the user breathing interface. For example, the display of relative pressure may include one or more bars of varying height or thickness indicative of the relative pressure at the user breathing interface (e.g., relative to a maximum pressure that may be provided by breathing apparatus 10). In one embodiment the contents of display 104 may be oriented based upon, at least in part, an orientation of housing 100, e.g., such that the contents of display 104 may always be oriented “right-side-up.” For example, when housing 100 is positioned on end, as shown in FIG. 6, the contents of display 104 may be oriented upwardly, as shown. However, when housing 100 is positioned on side (e.g., an orientation 90 degrees counterclockwise relative to the orientation depicted in FIG. 6), the contents of display 104 may be rotated 90 degrees counterclockwise relative to the orientation of the contents of display 104 shown in FIG. 6. Orientation of the contents of display 104 may be based upon, at least in part, a control signal provided, for example, by a three axis accelerometer.

In one or more embodiments, breathing apparatus 10 may include one or more light sensors. A brightness of contents of display 104, and/or one or more other illuminated indicators, may be varied based upon, at least in part, a detected ambient light level detected by the one or more light sensors.

As discussed above, breathing apparatus 10 may include one or more storage device, e.g., storage device 32. Storage device 32 may receive and store various information regarding the operation of breathing apparatus 10. Examples of information that may be received and stored may include, but is not limited to, operations pressure, changes in operation pressure, instances of detected snoring, and the like. Information stored on storage device 32 may be accessed and/or transferred to another computing device using any suitable interface, such as a universal serial bus interface, a wireless interface (e.g., WiFi interface, Bluetooth interface, or the like), an Ethernet interface, etc. In some embodiments, information stored on storage device 32 may be automatically, and/or responsive to a user input, transferred to a remote computing device, e.g., to allow analysis by service and/or medical professions.

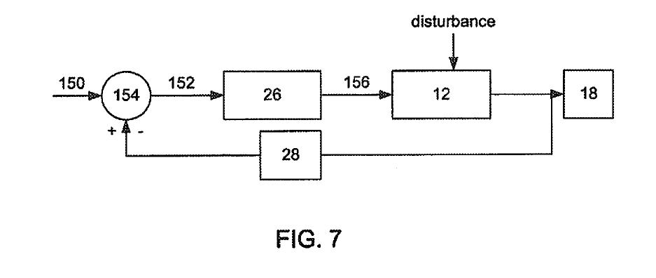

Referring to FIG. 7, and example of a feedback control system is schematically depicted. As discussed above, control system 16 may generally operate to maintain the pressure at user breathing interface 18 at a constant level, for example at a predetermined pressure in the range between about 2 cm H₂O and about 6 cm H₂O. As schematically depicted in FIG. 7, as desired pressure set point 150 may be received, for example, based upon a user setting provided via a user control such as push-turn control 102, which may allow the user to set a relative pressure level within the available range (e.g., between about 2 cm H₂O and about 6 cm H₂O in one embodiment). Controller 26 may receive an error signal 152 that may be calculated by error determining logic 154 as the difference between a pressure value at user breathing interface 18 determined by sensor 28 and desired pressure set point 150. In response to error signal 152, controller 26 (e.g., which may include a PI controller, as discussed above) may generate control signal 156 that may be fed to blower 12 (e.g., which may control a speed of motor 20) Blower 12 may generate a flow of breathing gas at a pressure based upon, at least in part, control signal 156. The resultant actual pressure at user breathing interface 18 may be measured by sensor 28 and fed back to error determining logic 154.

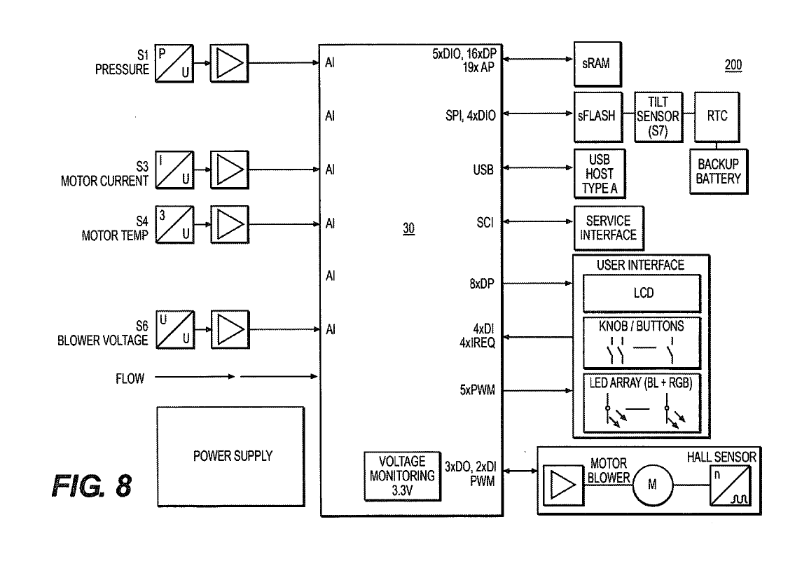

Referring to FIG. 8, an illustrative embodiment of at least a portion of a sensor and control system 200 is schematically depicted. For example, processor 30 receives an input from pressure sensor S1 as part a control system for controlling the pressure at user breathing interface 18. Processor 30 correspondingly provides an output signal for controlling the motor blower, e.g., which may generate the supply of breathing gas to be delivered to the user breathing interface 18. In the exemplary embodiment shown in FIG. 8, processor 30 receives an input from current sensor S3 that indicates a current demand by the blower motor, e.g., allowing the operation and/or condition of the motor to be monitored. As shown, processor 30 receives such exemplary inputs from various sensors, and provides a control signal to control a blower in accordance with such signals using, for example, common PI or a PID control.

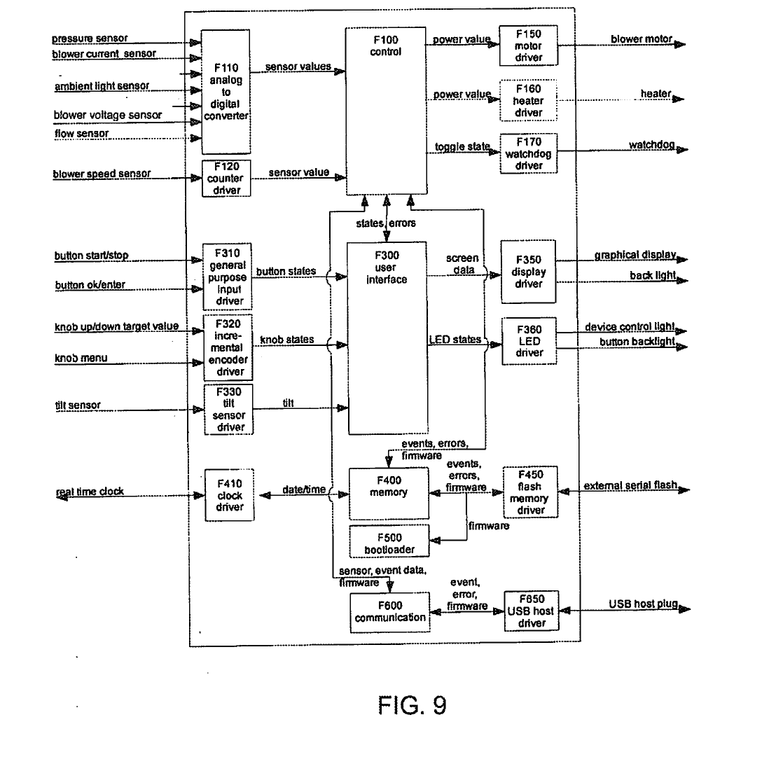

Referring also to FIG. 9, an embodiment of a modular control system 250 that may be implemented is schematically depicted. As shown, in the illustrated embodiment the modular control system 250 includes multiple control modules (e.g., control modules F100, F300, F400, F500, and F600) that receive various inputs and provide various control outputs. For example, control module F100 may receive, e.g., via analog to digital converter F110 and/or counter driver F120, one or more sensor inputs (e.g., pressure sensor input, blower current sensor input, voltage sensor input, flow sensor input, blower speed sensor input) relative to the operational condition of the blower and/or other elements of the breathing apparatus. Control module F100, provides one or more outputs to control the blower.

In an embodiment, control module F100 implements a blower motor control algorithm. As generally discussed above, the motor control algorithm can be implemented as a PI controller or PID controller. Via control module F100 such motor control algorithm can be implemented based upon, at least in part, sensor inputs (e.g., from a pressure and/or a flow sensor), which may be a control variable of the control circuit. Based upon, at least in part, the motor control algorithm and the sensor input, the motor may be controlled to achieve a stable pressure at the user breathing interface.

In the illustrative embodiment, memory allocation control module F400 controls the usage and allocation of memory associated with the breathing apparatus (e.g., of storage device 32), including what information may be stored in memory associated with the breathing apparatus. In this regard, the breathing apparatus may include one or more memory allocations and/or types of memory. For example, in an embodiment firmware may be stored on a flash memory.

According to one embodiment, program data memory may include static random access memory (SRAM). The SRAM memory may be utilized for stacks, buffers, operating system, drivers, character sets and double frame buffers for displays. Event memory, which may include, for example, snoring events, status messages (e.g., date, time of usage, etc.) may be stored on a non-volatile memory (e.g., flash memory, EEPROM, non-volatile SRAM, etc.), such that the event data may be maintained even during the loss of power to the breathing apparatus. In an illustrative embodiment, snoring event data may be stored for a predefined period of time (e.g., three months). Date, time and start volume data may be stored for each usage of the breathing apparatus may be stored during the lifetime of the breathing apparatus. Additionally, a time of any usage pauses, restarts, and end of usage data may also be stored Parameter data may also be stored in a non-volatile memory, such as flash memory of EEPROM. Parameter data may include serial number parameters that may be utilized by one or more programs executed by the breathing device.

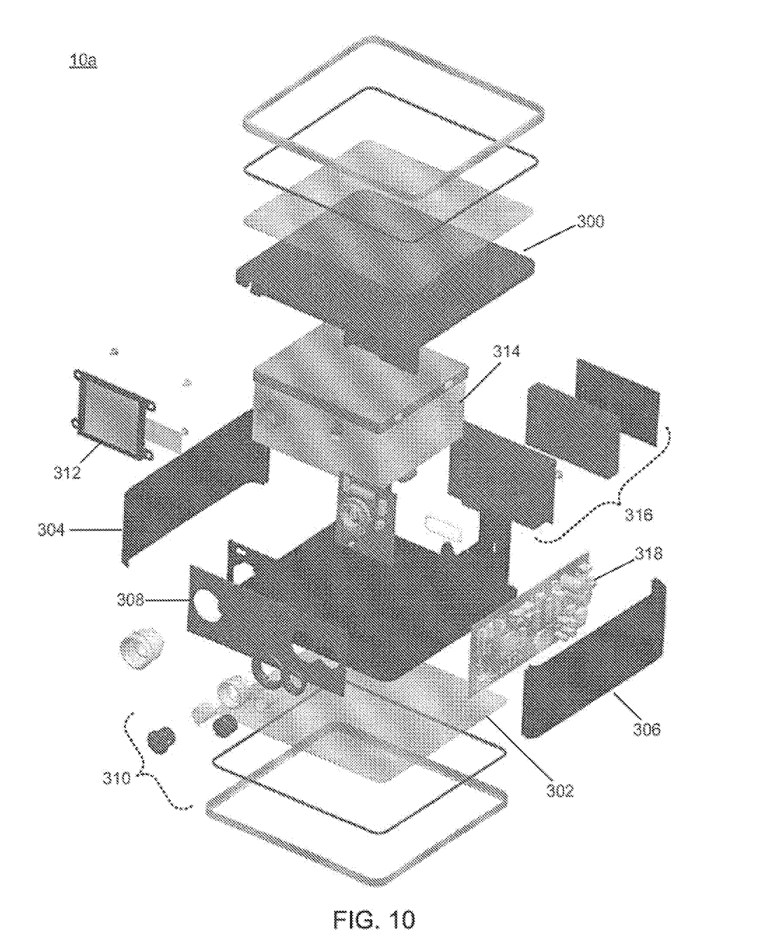

Referring to FIG. 10, there is shown an exploded view of an illustrative embodiment of breathing apparatus 10a. As shown, breathing apparatus 10a may generally include a housing assembly including top, bottom, and side panels, 300, 302, 304, and 306 respectively. Further, breathing apparatus 10a may include front panel 308 including one or more user interface controls 310 and display 312. Blower housing 314, which may contain the blower and provide acoustic and/or vibrational isolation of the blower, may be disposed at least partially within the housing. Filter assembly 316 may be associated with blower housing 314, e.g., for filtering air entering blower housing 314 via a blower intake (not shown). The filter can be structured to filter pollen Breathing apparatus 10a may further include one or more circuit boards (e.g., circuit board 318), which may include various control electronics, such as one or more control modules or controllers discussed hereinabove. In another variation, the breathing apparatus 10a may include a humidifier, which can be coupled with the filter. The humidifier could be configured to provide desired amounts of a drug or a fragrance.

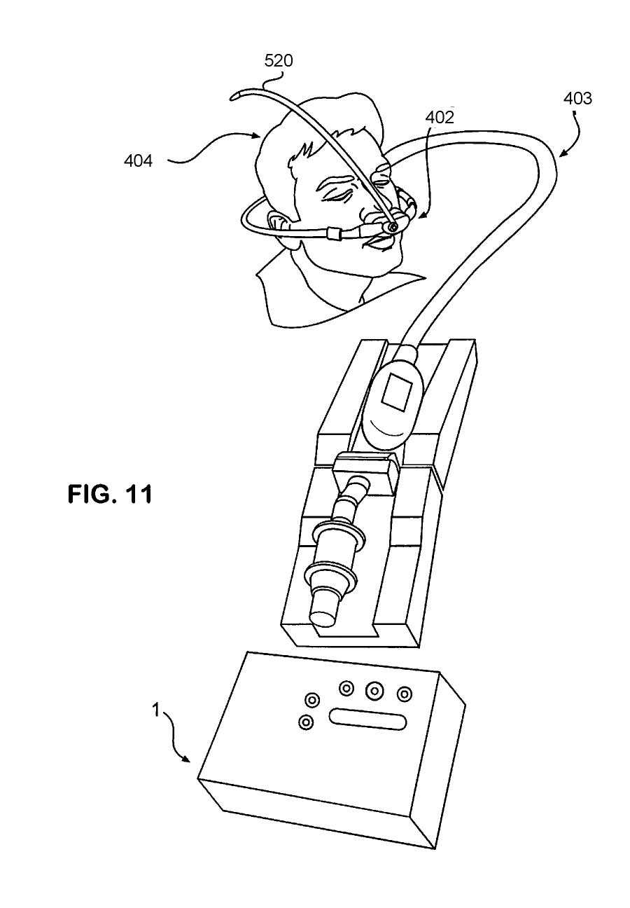

FIG. 11 is a schematic view of a general system that can employ a nasal interface in accordance with the present disclosure. Generally, an apparatus for treating snoring can included an air source 401, a nasal interface 402 and a connection 403, such as tubing, connecting the air source 401 and the nasal interface 402. The comfort of a user is important in the arrangement of a nasal interface. It is also important to try to minimize the amount of air flow that leaks out of the nasal interface. In other words, it is desirable to have an efficient coupling between the nasal interface and the user's nose so as to try to maximize the amount of air that is transferred between the nasal interface and the user's nose.

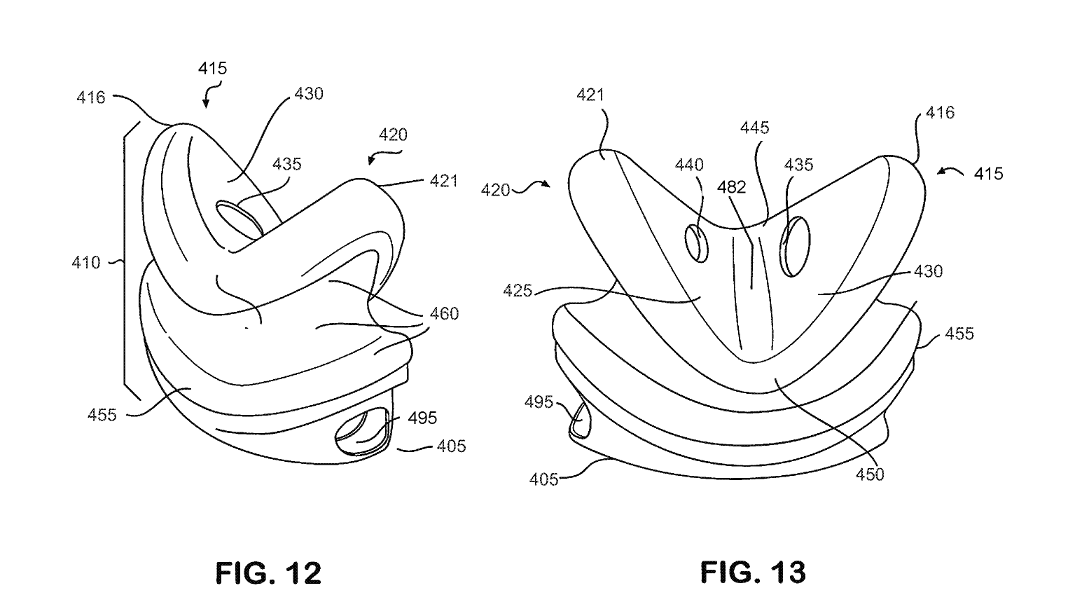

FIG. 12 is a perspective view of an illustrative embodiment of a nasal interface of the present disclosure. FIG. 13 is a front view of the illustrative embodiment of a nasal interface of the present disclosure shown in FIG. 12. Referring to FIGS. 12 and 13, the illustrative exemplary embodiment of a nasal interface includes a hard base portion 405 and a soft pad portion 410. The soft pad portion 410 can comprise a silicon pad, and the base portion 405 can be relatively harder than the soft pad portion 410. As illustrated in FIGS. 12 and 13, the soft pad portion 410 is unitary in nature and includes a winged shaped structure with wings 415 and 420. The wings 415 and 420 respectively include upper surfaces 425 and 430 with respective distal ends 416 and 421. The wings 415 and 420 respectively include therein air holes 435 and 440. These air holes are sized, positioned, and physically separated (spaced apart) on the upper surfaces 425 and 430 of wings 415 and 420 to locate the air holes in registry with a user's nostrils. An example of one such arrangement is shown in FIG. 18. As seen in FIG. 18, the upper surfaces 425 and 430 of the wings 415 and 420 engage the underside of a user's nose. As also described below, this allows air from the nasal interface to flow to the user's nostrils. In other embodiments, the upper surfaces 425 and 430 could include protrusions or other physically noticeable features about or near the air holes 435 and 440 to aid in positioning of the nasal interface with respect to a user's nostrils.

In an illustrative embodiment, the upper surfaces 425 and 430 may be positioned relative to each other at an angle of about 90° on the wings 415 and 420 of a nasal interface in accordance with the present disclosure. An exemplary vertex of a 90° angle corresponding to the included angle between the wings 415 and 420 is indicated by line 82 in FIG. 13. The selection of the angle is based upon ergonomic studies. Of course, in various embodiments the included angle may vary from about a 180° included angle to about a 45° included angle. The upper surfaces 425 and 430 may seal around the users nostrils as shown in FIG. 18. The upper surfaces 425 and 430 may be pressed smoothly against the user's nose by the systems pressure (e.g., mechanical pressure of the nasal interface as held against the user's nostrils as shown in FIG. 18) to form this seal.

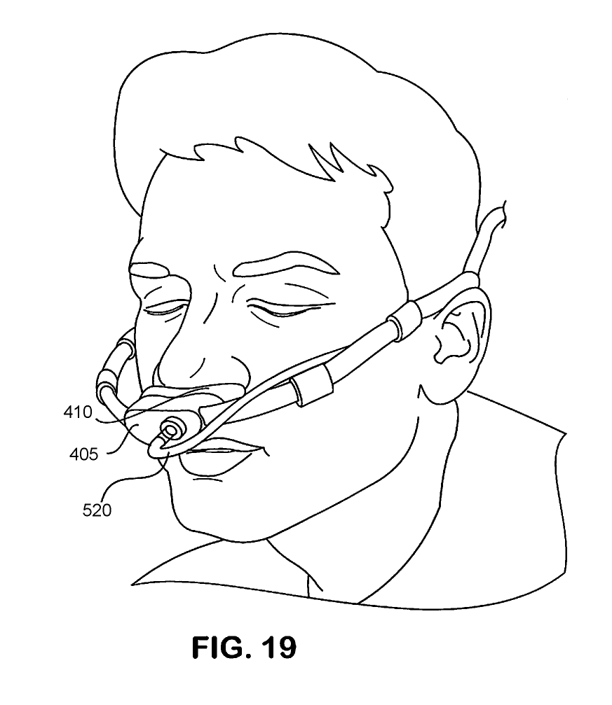

Referring to FIGS. 12 and 13, the upper surfaces 425 and 430 of wings 415 and 420 slope across a width of the pad portion 410 from a relatively higher position at point 445 to a relatively lower position at point 450. While not necessary to practicing the invention, this slope allows a nasal interface in accordance with the present disclosure to be positioned on a user's nose to conveniently accept tubing such as shown in FIGS. 18 and 19.

Referring to FIGS. 12 and 13, the soft pad portion 410 includes a lower border 455. In the illustrative embodiment, the lower border 455 aids in coupling the soft pad portion 410 to the base portion 405. The coupling can be any convenient coupling, such as a mechanical connection, an adhesive, welding, etc. In some embodiments, the lower border 455 may have a more or less round or oval cross section. The lower border 455 may be tensioned over an upper border of the base portion 410, which can be formed to fit using, for example, a key-lock type connection. The round shape may facilitate a tight sealing between the base and the soft pad 410 because of the tensile forces working radially toward to the center. Depending upon the use of the nasal interface and the type of coupling, the base portion 405 can be removable from the soft pad portion 410. Such removal facilitates cleaning the nasal interface or changing one of the base portion 405 or the soft pad portion 410.

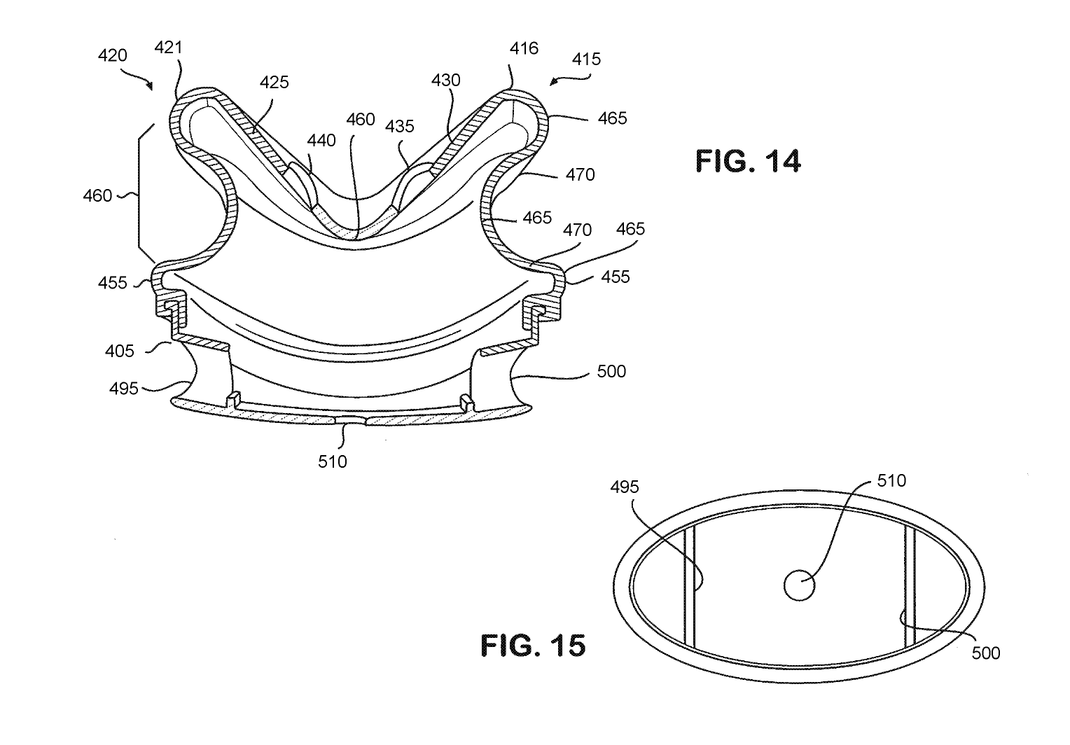

FIG. 14 is a cross sectional view of the illustrative embodiment show in FIGS. 12 and 13. In the illustrative embodiment shown in FIG. 14, the soft pad portion 410 includes a bellows 460. Bellows 460 may provide flexibility in height of the nasal interface and the angle at which the upper surfaces 425 and 430 reside with respect to a user's nostrils. The bellows 460 can function as a spring if there is pressure between the user's nose and the base portion 405.

The illustrated bellows includes vertical parts 465 (e.g., relative to the base) and horizontal parts 470 (e.g., relative to the base). The vertical parts 465 may give stability to the shape. The horizontal parts may allow a vertical movement of upper surfaces 425 and 430. This vertical movement may be for the whole soft pad portion 410, relative to the base portion 405. The vertical movement may be on one side of the pad, or both sides of the pad (including movement to different degrees on different sides). Thus, the bellows 460 allows the angle of the upper surfaces 425 and 430 relative to the base to change. This flexibility of the bellows and ability for the angle of the upper surfaces 425 and 430 to change may result in a self-sealing effect between a user's nostrils and the upper surfaces 425 and 430. While illustrated as extending around the nasal interface, the bellows 460 may extend only around a portion of the nasal interface.

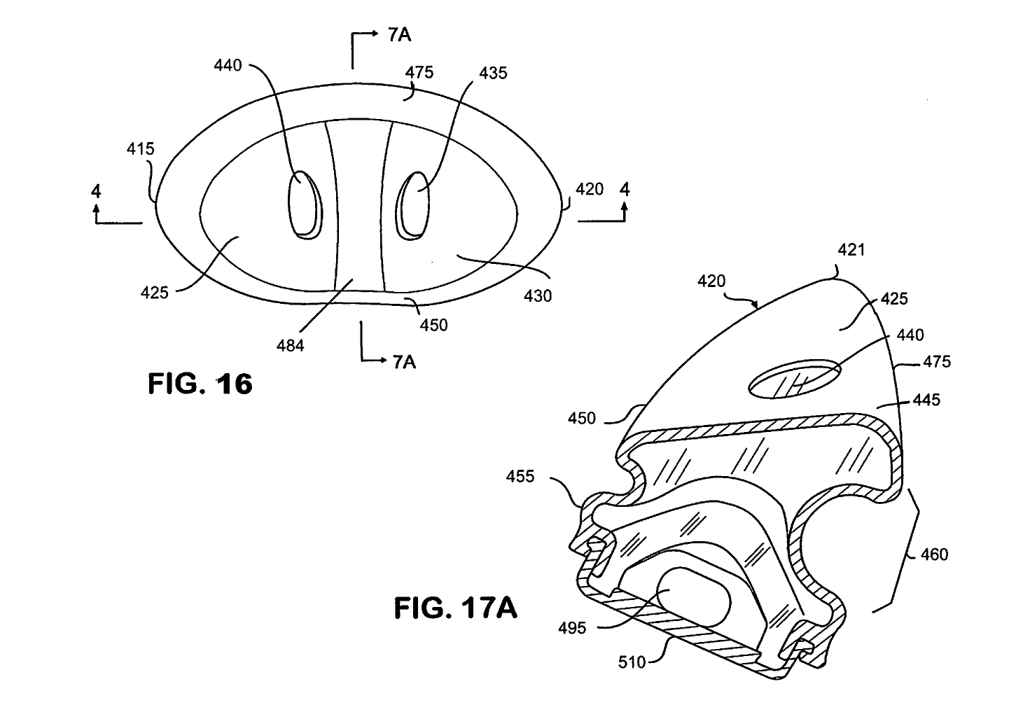

FIG. 16 is a partial top view of a soft pad portion 410 of a nasal interface in accordance with the present disclosure. The holes 435 and 440 in the upper surfaces 425 and 430 let the air into and out of a user's nostrils. The size and shape of these holes can be adjusted depending upon the desired flow and acoustic characteristics of a given design in accordance with the disclosure. While not shown in FIG. 16, the holes may have a cross section optimized for low pressure losses and low noise from the airflow through the holes. Also, in the illustrated embodiment, the holes 435 and 440 are near the edges between upper surfaces 425 and 430, and side surface 475. This position is shown in the illustrative embodiment because most people have nostrils beginning very near or directly above the user's upper lips. As further shown in the illustrated embodiment, as well as in FIGS. 12, 13, and 18, the wing portions and their upper surfaces 425, 430 are interconnected by way of a central connecting portion 484, which is interposed between the air holes 435, 440 of the wings 415, 420 and can seal against the central bottom strip (columella) of the user's nose during use. In the embodiment illustrated in FIG. 16, the connecting portion 484 of the pad 410 is devoid of any aperture.

Because a user's nostrils can begin directly from the upper lips there may not be a significant surface to seal the upper surfaces 425 and 430 to the user's nose. To address this, an illustrative embodiment may include side surface 475. The side surface 475 can aid in providing a seal between the nasal interface and the user. It can do so by the side surface 475 abutting and pressing against the user's upper lip.

The nasal interface, as shown in FIG. 19, may be positioned at an angle against the nose and upper lip of a user. In such an embodiment, the surfaces of the soft pad portion 410 have very soft and safe position. Because of the flexibility of the soft pad 410, such as provided by the bellows 460, the nasal interface can be adjusted relative to three planes: with respect to upper lips, and the two wings 415 and 420 with respect to a user's nostrils. This flexibility in positioning allows the position of the nasal interface to be highly defined. Gliding of the nasal interface may be reduced and/or prevented by this structural form. In addition tilting of the base portion 405 may prevent leakage due to the spring like function of the bellows 460 and the pressure the bellows 460 provides against a user's nose and the flexibility in angle of the upper surfaces 425 and 430 that the bellows 460 permits. Very low forces are needed with embodiments in accordance with the disclosure to effectively seal the nasal interface with a user's nostrils.

In the illustrated embodiment, the base portion 405 can be connected (e.g., glued, welded, snap or press fit, etc.) with one or more tubes as shown in FIG. 18. The illustrative embodiment shown in FIG. 18 utilizes two tubes 480 and 485, rather than one tube. The tubes connect to holes 495, 500 in the base portion 405.

The holes 495, 500 can be oriented in different angles. In one embodiment, the holes 495, 500 can be adjusted in every direction that is reasonable to guarantee a perfect seat of the nasal interface. While the illustrated embodiments depict two holes 495 and 500 for connecting to two tubes 480 and 485 on either side of the nasal interface, other arrangements may also be utilized. For example, one or more tubes may be connected at various locations on the nasal interface, such as the front of the nasal interface, or other suitable location.

The tubes 480, 485, in addition to supplying air to the nasal interface, may be utilized for holding the mask in the right position for maintaining connection to (e.g., sealing engagement with) the user's nose. In one embodiment, the tubes may come from a direction above the user's ear such as shown in FIG. 19. In other embodiments, a headgear arrangement not including the tubes (and/or in addition to the tubes) may be utilized for positioning the mask relative to the user.

Referring to FIGS. 14 and 15, the base portion 405 may have an additional hole 510. The size and shape of the hole 510 can be selected in accordance with the flow and acoustic characteristics of a given design in accordance with the disclosure. FIG. 15 illustrates a top view of the base portion 405 in accordance with an embodiment of the disclosure. The additional hole 510 can be, as shown in the illustrative embodiments, open to the environment (air outlet). In one embodiment, the hole 510 may be designed to allow airflow of about 20 l/min at 2 mbar overpressure inside the mask. This may reduce and/or prevent a CO₂-concentration to the user from being too high. Additionally, the hole 510 may be sized to provide an open breathing interface, such that the user may generally breathe ambient air entering through the hole. The ambient air may be supplemented with gasses (e.g., oxygen, etc.) supplied via the tube(s) (e.g., tubes 480, 485). As shown in FIGS. 18 and 19, an additional tube 520 can be used to measure the pressure within the nasal interface. It can also be used for pressure feedback to the air supply 401 shown in FIG. 11.

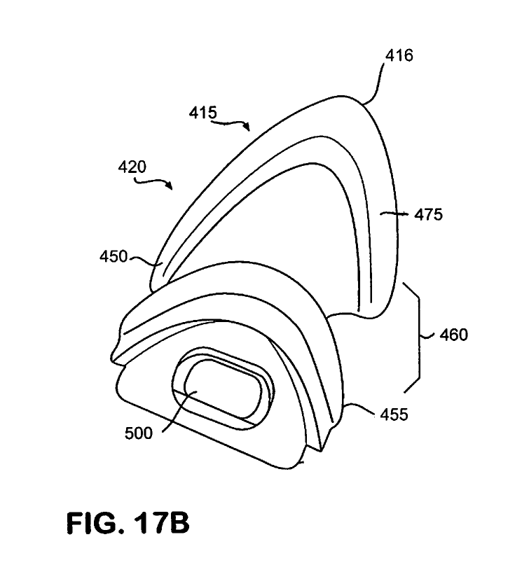

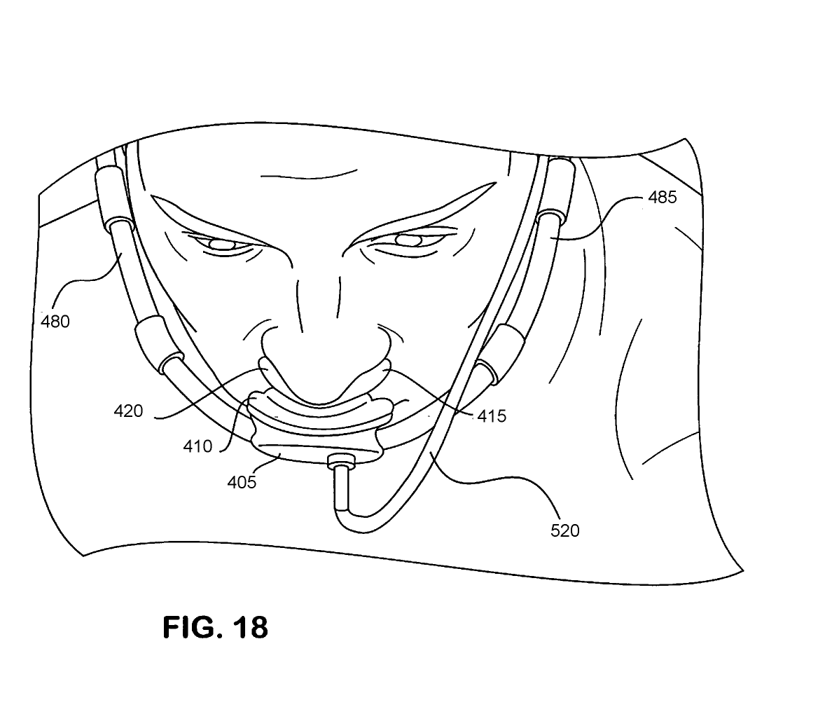

FIG. 17A is a cross-sectional view of a nasal interface in accordance with the present disclosure, viewed from the left side. FIG. 17B is a left side view of a nasal interface in accordance with the present disclosure. These views of an illustrative embodiment in accordance with the disclosure provide further illustration of the flexibility that the disclosed structure provides. FIG. 17A also illustrates the slope of the upper surface 425, as mentioned above.

For a nasal interface which fits a wide range of users, the nasal interface can have a relatively small dimension with respect to users' noses. In addition, the shape of the wings 425, 430, might be bigger depending upon the target size nose of users. As seen in FIGS. 12, 13, and 15, the base portion 405 has a relatively small dimension. The inner cross section, such as shown in FIG. 15, allows its overall shape to be selected for styling. The structure of the disclosed illustrative embodiments allows the base portion 405 to move sideward and upwards with reduced displacement of wings 425, 430 at the user's nose.

For the purpose of explanation various features and embodiments of a breathing apparatus and/or elements of a breathing apparatus have been described and depicted in the figures. It should be appreciated that the various features and embodiments may be susceptible to combination and substitution. For example, various features shown and/or described relative one or more embodiments may be combined with features shown and/or described relative to one or more other embodiments. Similarly, features described and/or shown relative to one or more embodiments may be substituted with features described and/or shown relative one or more other embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims.

That which is claimed is:

1. An apparatus comprising:
a blower configured to provide a supply of breathing gas;
a delivery tube configured to deliver the supply of breathing gas to a user breathing interface, the delivery tube having an inside diameter of about 15 mm or less;
the user breathing interface comprising
a base adapted to be connected to the delivery tube; and
a nasal pad including a lower border adapted to be connected to the base, the nasal pad including a pair of wing members positioned at an angle relative to one another, each wing member having at least one airflow aperture formed therein, each of the wing members being at least partially comprised of a hollow bladder fluidly communicating the delivery tube with the at least one airflow aperture, each wing member comprised of a substantially flat upper wall adapted to be placed in sealing contact with the underside of a user's nose, each upper wall being devoid of any protrusions adapted to enter the user's nostrils, each wing member also including a depending wall connecting the upper wall with the lower border; and
a control system configured to provide a control signal to the blower for controlling a pressure of the supply of breathing gas to between about 1 cm H₂O to about 6 cm H₂O at the user breathing interface, the control signal based upon, at least in part, one of a pressure and a flow rate of the supply of breathing gas at the user breathing interface.

2. The apparatus of claim 1, wherein the blower is configured to supply the breathing gas having a peak pressure of about 25 mbar at a flow rate of about 100 l/min at an outlet of the blower.

3. The apparatus according to claim 1, wherein the blower is configured to supply the breathing gas having a pressure of about 30 mbar and a flow rate of about 0 l/min at an outlet of the blower.

4. The apparatus according to claim 1, wherein the blower is configured to provide a flow rate acceleration of about 150 l/min/s over a flow rate range of about 0 l/min to about 100 l/min.

5. The apparatus according to claim 4, wherein the blower is configured to provide the flow rate acceleration of about 150 l/min/s over a pressure range of from about 0 mbar to about 25 mbar.

6. The apparatus according to claim 1, wherein the delivery tube includes a cross-sectional area adjacent the user breathing interface that is smaller than a cross-sectional area adjacent the blower.

7. The apparatus according to claim 1, wherein at least a portion of the delivery tube includes an exterior profile having an at least partially flat surface.

8. The apparatus according to claim 1, further comprising a pressure sensor coupled with the user breathing interface, the pressure sensor providing an output signal indicative of the pressure of the supply of breathing gas at the user breathing interface, wherein the control signal of the control system is based upon, at least in part, the output signal.

9. The apparatus according to claim 8, wherein the pressure sensor is coupled with the user breathing interface via a measurement lumen fluidly coupled with the pressure sensor and the user breathing interface.

10. The apparatus according to claim 9, wherein the delivery tube includes a multi-lumen tube including the measurement lumen and a breathing gas delivery lumen.

11. The apparatus according to claim 10, wherein a wall between the measurement lumen and the breathing gas delivery lumen is configured to de-couple pressure effects of the breathing gas in the breathing gas delivery lumen from the measurement lumen.

12. An apparatus comprising:
a blower assembly configured to provide a supply of breathing gas;
a delivery tube including a delivery lumen configured to deliver the supply of breathing gas to a user breathing interface, and a measurement lumen fluidly coupled to the user breathing interface, the delivery lumen having an inside diameter of about 15 mm or less;
a sensor fluidly coupled to the measurement lumen, the sensor configured to measure at least one of a pressure of breathing gas at the user breathing interface and a flow rate of breathing gas at the user breathing interface; and
a controller coupled to the blower for controlling an output characteristic of the breathing gas, based upon, at least in part, a measurement signal received from the sensor for controlling a pressure at the user breathing interface to between about 1 cm H₂O to about 6 cm H₂O;
wherein the user breathing interface comprises
a base adapted to be connected to the delivery tube; and
a nasal pad including a lower border adapted to be connected to the base, the nasal pad including a pair of wing members positioned at an angle relative to one another, each wing member having at least one airflow aperture formed therein, each of the wing members being at least partially comprised of a hollow bladder fluidly communicating the delivery tube with the at least one airflow aperture, each wing member comprised of a substantially flat upper wall adapted to be placed in sealing contact with the underside of a user's nose, each upper wall being devoid of any protrusions adapted to enter the user's nostrils, each wing member also including a depending wall connecting the upper wall with the lower border.

13. The apparatus according to claim 12, wherein a cross-sectional area of the delivery tube adjacent the blower assembly is greater than a cross-sectional area of the delivery tube adjacent the user breathing interface.

14. The apparatus according to claim 12, wherein the blower assembly has an acceleration of about 150 l/min/s over a flow rate from about 0 l/min to about 100 l/min.

15. The apparatus according to claim 12, further comprising a multi-lumen connector configured to couple the delivery lumen with the blower assembly and the measurement lumen with the sensor, the multi-lumen connector providing a rotationally symmetrical connection.

16. An apparatus comprising:
a housing assembly including a blower configured to provide a supply of breathing gas;
a user breathing interface configured to fluidly couple with an airway of a user, the user breathing interface comprising
a base adapted to be connected to the delivery tube; and
a nasal pad including a lower border adapted to be connected to the base, the nasal pad including a pair of wing members positioned at an angle relative to one another, each wing member having at least one airflow aperture formed therein, each of the wing members being at least partially comprised of a hollow bladder fluidly communicating the delivery tube with the at least one airflow aperture, each wing member comprised of a substantially flat upper wall adapted to be placed in sealing contact with the underside of a user's nose, each upper wall being devoid of any protrusions adapted to enter the user's nostrils, each wing member also including a depending wall connecting the upper wall with the lower border;
a supply tube configured to fluidly couple the blower with the user breathing interface, the supply tube having an inside diameter of between about 15 mm to about 5 mm; and
a control system for controlling the blower to provide a breathing gas pressure of between about 1 cm H₂O to about 6 cm H₂O at the user breathing interface.

17. The apparatus according to claim 16, wherein the control system includes a pressure sensor fluidly coupled with the user breathing interface.

18. The apparatus according to claim 17, wherein the supply tube includes a delivery lumen configured to fluidly couple the blower with the user breathing interface, and a measurement lumen configured to fluidly couple the sensor with the user breathing interface.

19. An apparatus comprising:
a blower configured to provide a supply of breathing gas based on a control signal;
a breathing interface configured to fluidly couple with an airway of a user;
a pressure sensor for providing a pressure signal;
a flow sensor in fluid communication with the supply of breathing gas provided by the blower for providing a flow signal;
a delivery tube configured to deliver the supply of breathing gas to the user breathing interface, the delivery tube having an inside diameter of about 15 mm or less and including a delivery lumen configured to fluidly couple the blower with the breathing interface, and a measurement lumen configured to fluidly couple the pressure sensor with the user breathing interface;
a control system configured to provide the control signal based on the pressure signal and the flow signal so as to control a pressure of the supply of breathing gas to between about 1 cm H₂O to about 6 cm H₂O at the user breathing interface, and a flow rate from about 0 l/min to about 100 l/min with a flow rate acceleration of about 150 l/min/s.

20. The apparatus of claim 19, wherein the user breathing interface comprises:
a base adapted to be connected to the delivery tube; and
a nasal pad including a lower border adapted to be connected to the base, the nasal pad including a pair of wing members positioned at an angle relative to one another, each wing member having at least one airflow aperture formed therein, each of the wing members being at least partially comprised of a hollow bladder fluidly communicating the delivery tube with the at least one airflow aperture, each wing member comprised of a substantially flat upper wall adapted to be placed in sealing contact with the underside of a user's nose, each upper wall being devoid of any protrusions adapted to enter the user's nostrils, each wing member also including a depending wall connecting the upper wall with the lower border.
